(19) United States
(12) Patent Application Publication
(10) Pub. No.: US 2026/0165840 A1
(43) Pub. Date: Jun. 18, 2026

(54) SYSTEMS AND DEVICES FOR REPAIR OF REGURGITANT VALVES

(71) Applicant: Edwards Lifesciences Corporation, Irvine, CA (US)

(72) Inventors: Yael Livneh, Palo Alto, CA (US); Brian Patrick Murphy, Irvine, CA (US); Bar Eytan-Vaisman, Gedera (IL); Eva Adriana de la Cruz Romito, Irvine, CA (US)

(21) Appl. No.: 19/535,567

(22) Filed: Feb. 10, 2026

Related U.S. Application Data
(63) Continuation of application No. PCT/US2024/041623, filed on Aug. 9, 2024.
(60) Provisional application No. 63/519,218, filed on Aug. 11, 2023.

(51) Int. Cl. A61F 2/24; A61L 27/20; A61L 27/22; A61L 27/24; A61L 29/06; A61M 25/00; A61M 25/01; A61M 25/06

(52) U.S. Cl. CPC A61F 2/2466; A61L 27/20; A61L 27/227; A61L 27/24; A61L 29/06; A61F 2210/0004; A61M 2025/0089; A61M 25/0194; A61M 2025/0681; A61M 2210/125

(57) ABSTRACT

Systems and devices for augmenting an annulus of a valve are provided. The systems and devices can be used for treating valve regurgitation. The systems and devices can include a transcatheter system to deliver the augmentation implant and/or filler to a valve. The systems and devices can provide transient, semi-permanent, and permanent augmentation of a valve annulus.

### CROSS REFERENCE TO RELATED APPLICATIONS

The current application is a continuation of International Patent Application No. PCT/US2024/041623, filed Aug. 9, 2024, which claims the benefit of and priority to U.S. Provisional Patent Application No. 63/519,218, filed Aug. 11, 2023, the disclosures of which are hereby incorporated by reference in their entireties for all purposes.

### BACKGROUND

The native heart valves (i.e., the aortic, pulmonary, tricuspid and mitral valves) serve critical functions in assuring the forward flow of an adequate supply of blood through the cardiovascular system. These heart valves can be rendered less effective by structural valvar abnormalities congenital malformations, inflammatory processes, infectious conditions, disease, and functional etiologies. Such damage to the valves can result in serious cardiovascular compromise or death. Treatment for such disorders can be done with the surgical repair or replacement of the valve during open heart surgery or with transvascular techniques for introducing and implanting prosthetic devices in a manner that is much less invasive than open heart surgery.

A healthy heart has a generally conical shape that tapers to a lower apex. The heart has four chambers: the left atrium, right atrium, left ventricle, and right ventricle. The left and right sides of the heart are separated by a wall generally referred to as the septum. The native tricuspid valve and mitral valve of the human heart connects the right atrium to the right ventricle and left atrium to the left ventricle, respectively. Each valve includes an annulus portion, which is an annular portion of the native valve tissue surrounding the valve orifice, and a pair leaflets (as referred to as cusps) that extend downward from the annulus into the ventricle.

When operating properly, the leaflets function together as a one-way valve to allow blood to flow only from the left atrium to the left ventricle. The left atrium receives oxygenated blood from the pulmonary veins. When the muscles of the left atrium contract and the left ventricle dilates, the oxygenated blood that is collected in the left atrium flows into the left ventricle. When the muscles of the left atrium relax and the muscles of the left ventricle contract, the increased blood pressure in the left ventricle urges the two leaflets together, thereby closing the one-way mitral valve so that blood cannot flow back to the left atrium and is instead expelled out of the left ventricle through the aortic valve. To prevent leaflets from prolapsing or flailing under pressure and folding back through the mitral annulus toward the left atrium, a plurality of fibrous cords called chordae tendineae tether the leaflets to papillary muscles.

One common medical complication with heart valves is valve regurgitation. Valve regurgitation is the failure of native valve to close properly, resulting in blood flowing back in a retrograde direction. Valve regurgitation is the most common form of valvular heart disease and can develop on any of the native heart valves, but especially common on the tricuspid and mitral valves. Valve regurgitation has different causes, including leaflet prolapse or flail, restricted leaflet motion (e.g., due to leaflet rigidity), and/or dysfunctional papillary muscles stretching.

Some techniques for treating leaflet valve regurgitation include surgical annuloplasty, annular reduction, and stitching or otherwise coupling portions of the native valve leaflets directly to one another. For example, mitral valve regurgitation can be treated by clipping the anterior and posterior leaflet together at the site of coaptation. Although some treatment options exist, there is a continuing need for improved devices and methods for treating valve regurgitation.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some aspects, the techniques described herein relate to a system for augmenting tissue, including: a transcatheter system, wherein the transcatheter system includes an outer sheath and a fluid injector or other means for fluid injection in fluidic connection with a biocompatible filler fluid; wherein the fluid injector or other means for fluid injection is capable of being distally advanced and proximately retracted in reference to the outer sheath.

In some aspects, the techniques described herein relate to a system, wherein the fluid injector or other means for fluid injection includes at least one of a needle and/or a cannula.

In some aspects, the techniques described herein relate to a system, wherein the fluid injector or other means for fluid injection is flexible.

In some aspects, the techniques described herein relate to a system, wherein the fluid injector or other means for fluid injection has a tip that is beveled, curved, or kinked.

In some aspects, the techniques described herein relate to a system, wherein the biocompatible filler fluid includes one of: saline, a solution including hyaluronic acid, a solution including collagen, a solution including elastin, or a solution including donated fat.

In some aspects, the techniques described herein relate to a method for augmenting annular tissue of a valve, including: advancing a transcatheter system to an annulus of a heart valve, wherein the transcatheter system includes an outer sheath and a fluid injector or other means for fluid injection in fluidic connection with a biocompatible filler fluid, wherein the means for fluid injection includes at least one of a needle and/or a cannula; and injecting the biocompatible filler fluid into the annulus.

In some aspects, the techniques described herein relate to a method, wherein the step of injecting the biocompatible filler fluid includes: repeatedly inserting the means of injection at a plurality of injection sites along the annulus; and depositing the biocompatible filler fluid at each of the plurality of injection sites.

In some aspects, the techniques described herein relate to a method, wherein the fluid injector or other means for injection is flexible and capable of tunneling within and around the annulus.

In some aspects, the techniques described herein relate to a method, wherein the step of injecting the biocompatible filler fluid includes: inserting the fluid injector or other means of injection within the annulus; tunneling the fluid injector or other means of injection through and around the annulus; and retracting the fluid injector or other means of injection back through and around the annulus, depositing the biocompatible filler fluid as the fluid injector or other means of injection is being retracted.

In some aspects, the techniques described herein relate to a method, wherein the step of injecting the biocompatible filler fluid includes: inserting the fluid injector or other means of injection within the annulus; depositing a portion of the biocompatible filler fluid at or near the insertion site; and advancing the fluid injector or other means of injection through and around the annulus, depositing the biocompatible filler fluid as the means of injection is advanced.

In some aspects, the techniques described herein relate to a system for permanently or semi-permanently augmenting tissue, including: a transcatheter system, wherein the transcatheter system includes an outer sheath and a compacted capsule in fluidic connection with a pump, wherein the compacted capsule is capable of being unfurled and elongated by filling the capsule with a filler; wherein the compacted capsule is at or near a distal end of the transcatheter system within the outer sheath.

In some aspects, the techniques described herein relate to a system, wherein the transcatheter system further includes a tool for creating an incision or nick in tissue, wherein the tool is at or near a distal end of the transcatheter system within the outer sheath.

In some aspects, the techniques described herein relate to a system, wherein the capsule includes a nonbiodegradable biocompatible material.

In some aspects, the techniques described herein relate to a system, wherein the capsule includes at least one of the following materials: poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), or a siloxane.

In some aspects, the techniques described herein relate to a system, wherein the capsule includes a biodegradable biocompatible material.

In some aspects, the techniques described herein relate to a system, wherein the capsule includes at least one of the following materials: poly(lactic-co-glycolic acid) (PGLA), poly (β-hydroxybutyrate-co-β-hydroxy valerate) (PHBV), polyhydroxy butyrate (PHB), or polycaprolactone (PCL).

In some aspects, the techniques described herein relate to a system, wherein the filler includes: silicone gel, saline, a solution including hyaluronic acid, a solution including collagen, a solution including elastin, or a solution including donated fat.

In some aspects, the techniques described herein relate to a system, wherein the transcatheter system further includes a removable nosecone at the distal end of the transcatheter system.

In some aspects, the techniques described herein relate to a system, wherein the pump is located within an outer catheter.

In some aspects, the techniques described herein relate to a system, wherein the pump is located at a proximal end of the transcatheter system.

In some aspects, the techniques described herein relate to a method for augmenting annular tissue of a valve, including: advancing a transcatheter system to an annulus of a heart valve, wherein the transcatheter system includes a capsule that is compacted and is in fluidic connection with a pump; creating an incision within the annulus; and elongating the capsule within the incision and through and around the annulus by filling the capsule with a filler via the pump.

In some aspects, the techniques described herein relate to a method, wherein the step of elongating the capsule includes elongating the capsule through softer fatty tissue that encircles the annulus.

In some aspects, the techniques described herein relate to a method further including: prior to elongating the capsule, inserting a tool within the incision and advancing the tool through and around the annulus to create a track.

In some aspects, the techniques described herein relate to a method, wherein the capsule includes a nonbiodegradable biocompatible material.

In some aspects, the techniques described herein relate to a method, wherein the capsule includes at least one of the following materials: poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), or a siloxane.

In some aspects, the techniques described herein relate to a method, wherein the capsule includes a biodegradable biocompatible material.

In some aspects, the techniques described herein relate to a method, wherein the capsule includes at least one of the following materials: poly(lactic-co-glycolic acid) (PGLA), poly (β-hydroxybutyrate-co-β-hydroxy valerate) (PHBV), polyhydroxy butyrate (PHB), or polycaprolactone (PCL).

In some aspects, the techniques described herein relate to a method, wherein the filler includes: silicone gel, saline, a solution including hyaluronic acid, a solution including collagen, a solution including elastin, or a solution including donated fat.

In some aspects, the techniques described herein relate to a method, wherein the step of elongating the capsule includes elongating the capsule through at least ⅛ of the annular circumference.

In some aspects, the techniques described herein relate to a method further including: sealing the capsule.

In some aspects, the techniques described herein relate to a system for installing an implant in tissue, including: a transcatheter system, wherein the transcatheter system includes a tissue excision tool or other means for excising tissue, an implant, and a delivery tool or other means for installing the implant within tissue.

In some aspects, the techniques described herein relate to a system, wherein the tissue excision tool or other means for excising tissue is a sharp-edged tool.

In some aspects, the techniques described herein relate to a system, wherein the sharp-edged tool is a scalpel or a tissue shaver.

In some aspects, the techniques described herein relate to a system, wherein the tissue excision tool or other means for excising tissue is a tissue ablation tool.

In some aspects, the techniques described herein relate to a system, wherein the tissue ablation tool uses heat ablation, radiofrequency ablation, cryoablation, or electroablation.

In some aspects, the techniques described herein relate to a system, wherein the implant includes a biocompatible polymer that is capable of being delivered as a liquid or gel.

In some aspects, the techniques described herein relate to a system, wherein the biocompatible polymer is capable of curing, hardening, or solidifying.

In some aspects, the techniques described herein relate to a system, wherein the biocompatible polymer is a hydrogel.

In some aspects, the techniques described herein relate to a system, wherein the implant includes a plurality of biocompatible beads.

In some aspects, the techniques described herein relate to a system, wherein each biocompatible bead is a silica-based or hydrogel-based.

In some aspects, the techniques described herein relate to a system, wherein the implant includes an embolization coil.

In some aspects, the techniques described herein relate to a system, wherein the embolization coil includes a biocompatible metal with linear elasticity.

In some aspects, the techniques described herein relate to a system, wherein the implant includes an expandable stent.

In some aspects, the techniques described herein relate to a system, wherein the expandable stent is self-expanding.

In some aspects, the techniques described herein relate to a system, wherein the expandable stent includes a substantially impermeable cover.

In some aspects, the techniques described herein relate to a method for installing an implant in an annulus of a heart valve, including: advancing a transcatheter system to an annulus of a heart valve, wherein the transcatheter system includes a tissue excision tool or other means for excising tissue, an implant, and a delivery tool or other means for installing the implant within tissue; excising tissue, using the tissue excision tool or other means for excising tissue, from the annulus to form a cavity therein; and installing the implant in the cavity.

In some aspects, the techniques described herein relate to a method, wherein the tissue excision tool or other means for excising tissue is a sharp-edged tool.

In some aspects, the techniques described herein relate to a method, wherein the sharp-edged tool is a scalpel or a tissue shaver.

In some aspects, the techniques described herein relate to a method, wherein the tissue excision tool or other means for excising tissue is a tissue ablation tool.

In some aspects, the techniques described herein relate to a method, wherein the tissue ablation tool uses heat ablation, radiofrequency ablation, cryoablation, or electroablation.

In some aspects, the techniques described herein relate to a method, wherein the implant includes a biocompatible polymer capable of being delivered as a liquid or gel, and wherein the step of installing the implant includes depositing the biocompatible polymer into the cavity.

In some aspects, the techniques described herein relate to a method, wherein the biocompatible polymer is capable of curing, hardening, or solidifying.

In some aspects, the techniques described herein relate to a method, wherein the biocompatible polymer is a hydrogel.

In some aspects, the techniques described herein relate to a method, wherein the implant includes a plurality of biocompatible beads, and wherein the step of installing the implant includes depositing the plurality of biocompatible beads into the cavity.

In some aspects, the techniques described herein relate to a method, wherein each biocompatible bead is a silica-based or hydrogel-based.

In some aspects, the techniques described herein relate to a method, wherein the implant includes an embolization coil and wherein the step of installing the implant includes depositing the embolization coil into the cavity.

In some aspects, the techniques described herein relate to a method, wherein the embolization coil includes a biocompatible metal with linear elasticity.

In some aspects, the techniques described herein relate to a method, wherein the implant includes an expandable stent, and wherein the step of installing the implant includes expanding the expandable stent within the cavity.

In some aspects, the techniques described herein relate to a method, wherein the expandable stent is self-expanding.

In some aspects, the techniques described herein relate to a method, wherein the expandable stent includes a substantially impermeable cover.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

### BRIEF DESCRIPTION OF THE DRAWINGS

The description and claims will be more fully understood with reference to the following figures, which are presented as examples of the disclosure and should not be construed as a complete recitation of the scope of the disclosure.

FIGS. 1A and 1B provide views of a tricuspid valve.

FIGS. 1C and 1D provide views of an example of a regurgitant tricuspid valve.

FIGS. 2A and 2B provide views of a mitral valve.

FIGS. 2C and 2D provide views of an example of a regurgitant mitral valve.

FIGS. 3A to 3F provide views of an example of a system and method for treatment of a regurgitant valve via annular augmentation.

FIGS. 4A to 4C provide views of an example of a catheter system for treatment of a regurgitant valve using an encapsulated filler.

FIGS. 5A to 5F provide views of an example of a system and method for treatment of a regurgitant valve via encapsulated annular augmentation.

FIGS. 6A to 6I provide views of an example of a system and method for treatment of a regurgitant valve using an annular implant.

### DETAILED DESCRIPTION

The current disclosure details systems and devices for repairing a regurgitant valve. The systems and devices can utilize a catheter design for minimal invasiveness and/or travel through the circulatory within the body. Accordingly, systems and devices can comprise a catheter that provides way to translocate tools for repairing a regurgitant valve by augmenting the annulus of the valve. Augmentation of the annulus will yield an expanded mass within the annular tissue that will push the leaflets more towards the center of the valve, promoting proper coaptation. The systems, devices, and methods described herein can be utilized as an independent treatment or utilized in a combination with any other compatible treatment for valve regurgitation.

Some of the systems and devices of the current disclosure are directed to augmenting the annulus of a valve, which can comprise a catheter-based filler delivery system for annular augmentation. These systems and devices can be utilized to reach a valve annulus via a catheter delivery system comprising a needle (or cannula, hypotube, fluid line, etc.) for delivering augmenting filler. In some implementations, the needle (or cannula, hypotube, fluid line, etc.) is inserted into multiple locations within the annulus to deposit filler at each location. In some implementations, the needle (or cannula, hypotube, fluid line, etc.) is inserted into an initial point in the annulus to deposit an initial amount of filler to create a track. The needle (or cannula, hypotube, fluid line, etc.) can follow the track depositing more filler along the way to generate more track for the needle (or cannula, hypotube, fluid line, etc.) to follow. In some implementations, the needle (or cannula, hypotube, fluid line, etc.) is inserted into the annulus and follows a track. The needle (or cannula, hypotube, fluid line, etc.) is then retracted back through the track, depositing filler as it is retracted.

Some of the systems and devices of the current disclosure are directed to permanent (or semi-permanent) augmentation of the annulus of a valve, which can comprise a catheter-based encapsulated filler delivery system for annular augmentation. These systems and devices can be utilized to reach a valve annulus via a catheter delivery system comprising a compacted capsule capable of being unfurled, elongated and filled. In some implementations, a tissue tunneling tool is utilized to tunnel a track along the annulus. Upon completion of a tracked tunnel, the compacted capsule is unfurled and elongated within the tunnel. In some implementations, a compacted capsule is powered to tunnel through and elongate along the annulus. In some implementations, the capsule is filled with a filler as the capsule is elongated. In some implementations, the capsule is filled with a filler after elongation of the capsule. In some instances, a pump is utilized to fill the capsule. In some instances, the pump powers (or assists) the elongation of the capsule.

Some of the systems and devices of the current disclosure are directed to implants of the annulus of a valve, which can comprise a catheter-based ablation of tissue and catheter-based delivery of implants. These systems and devices can be utilized to reach a valve annulus via a catheter ablation system comprising cavity forming tool or other means to create a cavity within the annulus and a catheter delivery system to deliver implants to fill the cavity and augment the annulus. In some implementations, a set of one or more cavities are created along the valve annulus. Each cavity is filled with an implant filler to augment the annulus. In some implementations, the implant filler is a glue. In some implementations, the implant filler is a plurality of beads. In some implementations, the implant filler is a plurality of embolization coils. In some implementations, the implant filler is an expandable stent.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of annular augmentation systems and components thereof are disclosed herein, and any combination of these examples can be made unless specifically excluded. For example, an augmenting filler (transient or permanent) can be combined with annular implants, even if a specific combination is not explicitly described. Likewise, the different constructions and features of annular augmentation systems can be mixed and matched, such as by combining the various components of the systems, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

Further, the techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards (or passed) a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices for tissue repair are utilized for the purpose of performing a procedure within a recipient. Recipients include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.)

For the purpose of understanding leaflet prolapse, the tricuspid valve and mitral valve are utilized as examples in the following figures and description. It is to be understood that valve regurgitation can occur in any heart valve and thus reference to the tricuspid valve and mitral valve and treatments thereof are provided as an example of any of the valves, including the pulmonary valve or the aortic valve. In other words, any of the methods, devices, or description herein related to treatment of a particular valve can be utilized and/or adapted to be utilized on the tricuspid valve, mitral valve, pulmonary valve, or aortic valve.

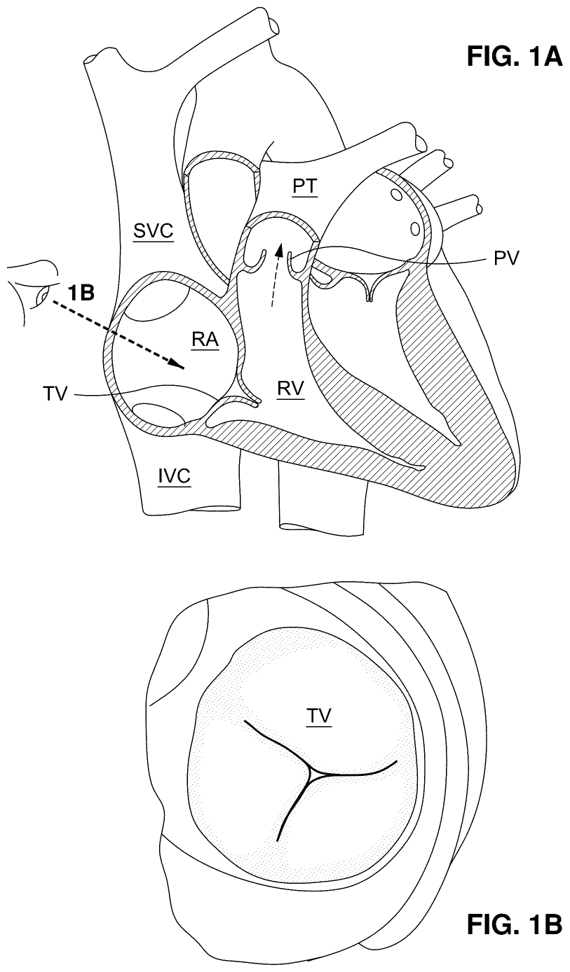

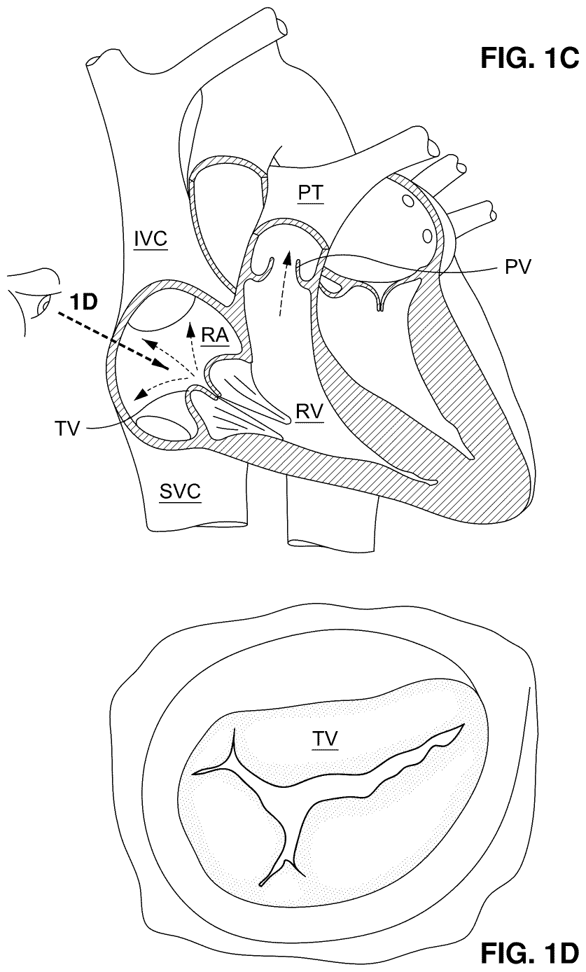

FIG. 1A is a coronal-plane view of the heart sectioning through the coaptation area of the mitral valve and FIG. 1B is a traverse-plane view within the right atrium superior to the tricuspid valve. The right ventricle (RV) is separated from the right atrium (RA) via the tricuspid valve (TV). Each of the four valves of the heart has flexible leaflets extending inward across the respective orifices that come together or “coapt” in the flowstream to form the one-way, fluid-occluding surfaces. Accordingly, referring back to the right chambers, deoxygenated blood is returned to the right atrium from the inferior vena cava (IVC) and superior vena cava (SVC) and then transferred across the tricuspid valve into the right ventricle. During systole, the right ventricle contracts to pump the deoxygenated through the pulmonary valve (PV) and into the pulmonary trunk (PT) and lungs to be oxygenated.

FIGS. 1C and 1D provide an example of a regurgitant tricuspid valve. Regurgitation can occur when the set tricuspid leaflets are unable to coapt during systole, i.e., when the right ventricle contracts. The ventricle contraction results in the blood being pumped forward into the pulmonary trunk and retrograde back across the tricuspid valve into the right atrium (FIG. 1C). The retrograde flow decreases the amount of forward flow into the lungs. To compensate for the lack of forward flow into the lungs, the right ventricle can expand and pump harder, which can cause the ventricle to weaken overtime and result in heart failure. Furthermore, tricuspid valve regurgitation can cause fatigue, arrhythmias, pulsing in the neck, and shortness of breath.

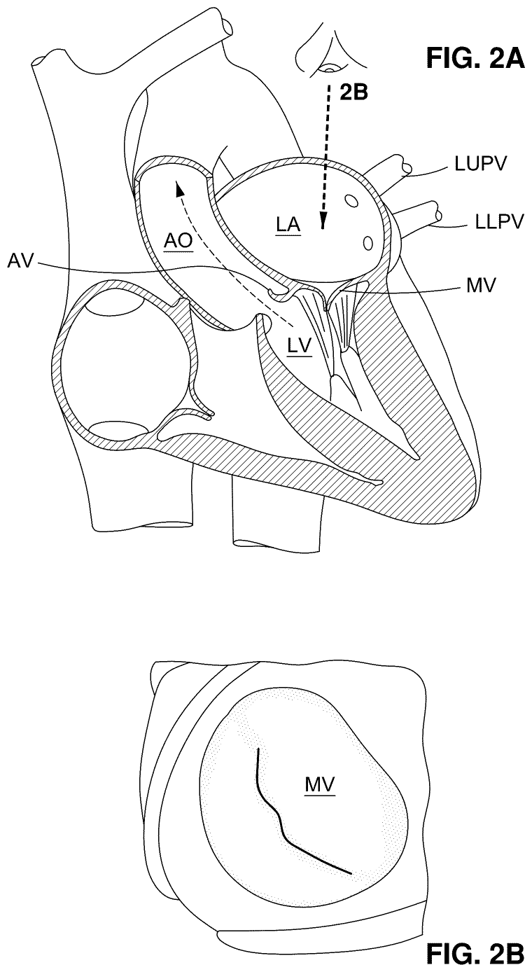

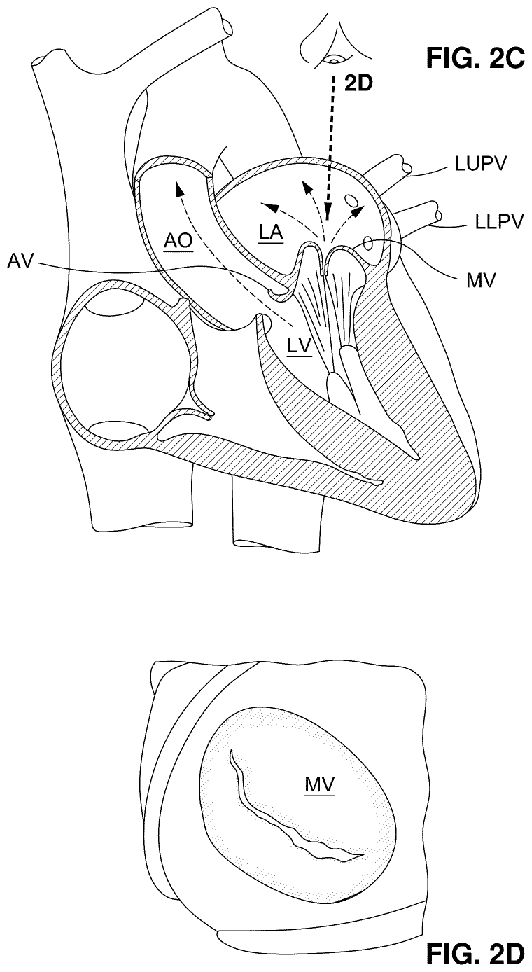

FIG. 2A is a coronal-plane view of the heart sectioning through the coaptation area of the mitral valve and FIG. 2B is a traverse-plane view within the left atrium superior to the mitral valve. The left ventricle (LV) is separated from the left atrium (LA) via the mitral valve (MV). Each of the four valves of the heart has flexible leaflets extending inward across the respective orifices that come together or “coapt” in the flowstream to form the one-way, fluid-occluding surfaces. Accordingly, referring back to the left chambers, oxygenated blood is brought to the left atrium from the pulmonary veins (LUPV/LLPV) and then transferred across the mitral valve into the left ventricle. The left ventricle pumps the oxygenated passing through the aortic valve (AV), into the aorta (AO), and throughout the body.

FIGS. 2C and 2D provide an example of a regurgitant mitral valve. Regurgitation can occur when the two mitral leaflets are unable to coapt during systole, i.e., when the left ventricle contracts. The ventricle contraction results in the blood being pumped forward into the aorta and retrograde back across the mitral valve into the left atrium (FIG. 2C). The retrograde flow decreases the amount of forward flow throughout the body. To compensate for the lack of forward flow throughout the body, the left ventricle can expand and pump harder, which can cause the ventricle to weaken overtime, and resulting in atrial fibrillation, pulmonary hypertension, and/or congestive heart failure. Furthermore, mitral valve regurgitation can cause a heart murmur, arrhythmias, shortness of breath, palpitations, and edema in the extremities.

Systems and devices for augmentation of the annulus comprise a catheter system such that it can be utilized within minimally invasive procedures and/or traverse through the circulatory system to reach the site of procedure. The systems and devices can comprise a delivery catheter for delivery of filler into the annulus of a valve. A delivery catheter systems and devices can comprise a fluid injector or other means for injection, such as a means for injecting filler into the annulus.

The systems and devices for augmentation can be utilized on the annulus of any of the four valves of the heart, including the mitral valve, the tricuspid valve, the pulmonary valve, and the aortic valve. Any transcatheter approach can be utilized to reach the valve to be treated, such as (for example) a transfemoral, subclavian, transapical, transjugular, or transaortic approach.

In some implementations, the fluid injector or other means for injection comprises one or more of a needle, a syringe, a cannula, a fluid line, a hypotube, a pump, a balloon, a hydraulic system, a power injector, a motor, etc., but any means for delivery a fluid into a tissue can be utilized. In some implementations, the fluid injector or means for injection (e.g., all or only a portion thereof), whether a needle, cannula, or otherwise, is flexible or comprises a flexible portion, which can help traverse the circulatory system via the catheter and/or tunnel through and around the valve annulus

In some implementations, the fluid injector or means for injection (e.g., all or only a portion thereof), whether a needle, cannula, or otherwise, can have a length to tunnel through a portion of the annulus. In some implementations, the fluid injector or means for injection has length sufficient to tunnel through at least ¾ of the annular circumference, the fluid injector or means for injection has length sufficient to tunnel through at least ⅔ of the annular circumference, the fluid injector or means for injection has length sufficient to tunnel through at least ½ of the annular circumference, the fluid injector or means for injection has length sufficient to tunnel through at least ⅓ of the annular circumference, the fluid injector or means for injection has length sufficient to tunnel through at least ¼ of the annular circumference, and/or the fluid injector or means for injection has length sufficient to tunnel through at least ⅛ of the annular circumference.

In some implementations, the gauge of the fluid injector or means of injection, whether a needle, cannula, or otherwise, is between 18 and 30. In various implementations, the gauge of the fluid injector or means of injection is 18, the gauge of the fluid injector or means of injection is 19, the gauge of the fluid injector or means of injection is 20, the gauge of the fluid injector or means of injection is 21, the gauge of the fluid injector or means of injection is 22, the gauge of the fluid injector or means of injection is 23, the gauge of the fluid injector or means of injection is 24, the gauge of the fluid injector or means of injection is 25, the gauge of the fluid injector or means of injection is 26, the gauge of the fluid injector or means of injection is 27, the gauge of the fluid injector or means of injection is 28, the gauge of the fluid injector or means of injection is 29, or the gauge of the means of injection is 30.

In some implementations, the fluid injector or means for injection, whether a needle, cannula, or otherwise, includes a beveled and/or curved/kinked tip such that the needle deflects as it tunnels through tissue. The bevel, curve, contour and gauge of the fluid injector or means for injection can be selected such that the fluid injector or means for injection can be steered in a desired pathway along the annulus. Examples of tips that can be utilized include (but are not limited to) Quincke tip, Chiba tip, Tuohy tip, Hustead tip, and Whitacre-point tip. The fluid injector or means for injection can further include a flexible shaft. The fluid injector or means for injection can further include a flexure joint such that when the fluid injector or means for injection is twisted, the bevel tip can be reoriented to further control the steerability of the needle (see, e.g., Swaney et al., IEEE Trans Biomed Eng. 2013; 60(4):906-9, the disclosure of which is herein incorporated by reference.) The fluid injector or means for injection can further telescoping shaft, a controlled articulating tip, actuation cables, an actuated hinge, or magnets for active needle steering (See, e.g., Gilbert et al., EEE Trans Robot. 2015; 31(2):246-258; Adebar et al., IEEE Trans Biomed Eng. 2016; 63(6):1167-77; van de Berg et al., Med Eng Phys. 2015; 37(6):617-22; and Ilami et al., Sci Rep. 2020; 10(1):2500; the disclosures which are each herein incorporated by reference).

In some implementations, the fluid injector or means for injection, whether a needle, cannula, or otherwise, is in fluidic connection with an inner catheter for the storage and transfer of filler. In some implementations, the inner catheter can comprise a means for manual injection. For example, in some implementations, the inner catheter can be a syringe (or is syringe-like) comprising a plunger for the expulsion of fluid through and out of the fluid injector or means for injection. Other means for manually pushing fluid out of an inner catheter can also be utilized. In some implementations, the inner catheter can be in connection with a pump that can provide pressurized fluid (e.g., filler, saline, carbon dioxide gas) to push the filler through and out the fluid injector or means for injection.

Any biocompatible filler can be utilized for annular augmentation. Examples of biocompatible fillers include (but are not limited to) saline, a solution comprising hyaluronic acid, a solution comprising collagen, a solution comprising elastin, a solution comprising donated fat, a and combinations thereof. In one example, donated fat can be removed from a location within the patient's body (e.g., hips, thighs, abdomen) and transferred to the catheter system for injection. The selection of various biocompatible fillers can depend on the needs of the patient and the ability of the filler to be retained within the site of injection. For instance, donated fat can retain within the injection site up to five years after injection, hyaluronic acid can retain within the injection site from six months to a year, and saline retain within the injection site for a shorter period of time.

In some implementations, the delivery catheter system extends proximally to a control system, as is appreciated in the art of transcatheter procedures. A clinician can utilize the control system to advance the transcatheter system through the body to the site where the procedure is to be performed. Further, the control system can advance and retract the fluid injector or means for injection in the distal and proximal direction in reference to an outer sheath. In some implementations, the control system can be utilized to control the steerability of the fluid injector or means for injection. In some implementations, the control system can further be utilized to control the injection of filler.

When at the valve annulus, various injection procedures can be performed. In some situations, the fluid injector or means for injection, whether a needle, cannula, or otherwise, can be repeatedly inserted at a plurality of injection sites along the annulus, depositing filler at each injection site. The injections can be repeated as necessary such that the annulus (or a portion thereof) is augmented with the filler and the leaflets of the valve can better coapt.

In some situations, the fluid injector or means for injection, whether a needle, cannula, or otherwise, is inserted at a location at or near the annulus and the fluid injector or means for injection is tunneled and steered along annulus. The fluid injector or means for injection can be inserted and tunneled up to a desired amount at which point filler is deposited. The fluid injector or means for injection can be retracted back through tissue, depositing filler along the way until the tip of the fluid injector or means for injection is removed from the tissue. The injections can be repeated as necessary such that the annulus (or a portion thereof) is augmented with the filler and the leaflets of the valve can better coapt.

In some situations, the fluid injector or means for injection, whether a needle, cannula, or otherwise, is inserted at a location at or near the annulus at which point a portion of the filler is deposited to augment and expand the annulus at the injection site and surrounding area. The fluid injector or means for injection can be further advanced through the augmented and expanded space within the annular tissue, which can provide a pathway to help the means of tissue tunnel through the annular tissue. As the fluid injector or means for injection advances along the annulus, filler is further deposited to further augment and expand the annulus and provide a pathway for further advancement. The fluid injector or means for injection can be advanced and can deposit filler up until a desired amount at which point the fluid injector or means for injection can be retracted along the augmented pathway until it is retracted. The injections can be repeated as necessary such that the annulus (or a portion thereof) is augmented with the filler and the leaflets of the valve can better coapt.

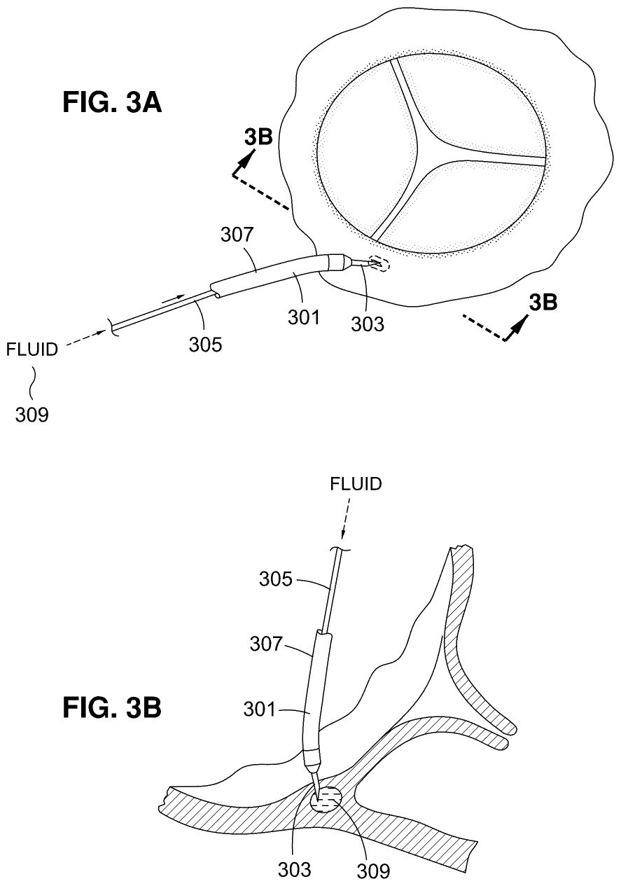

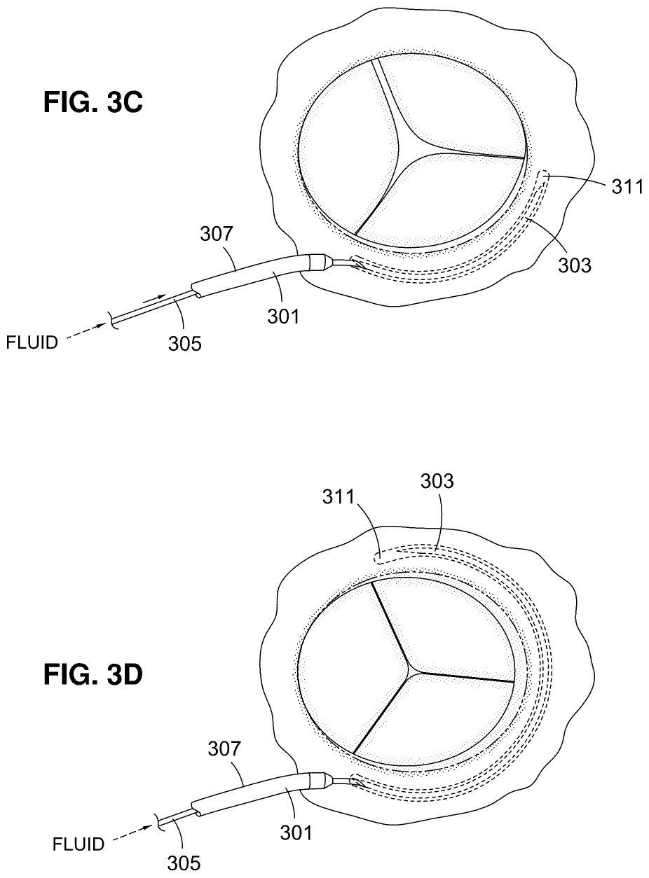

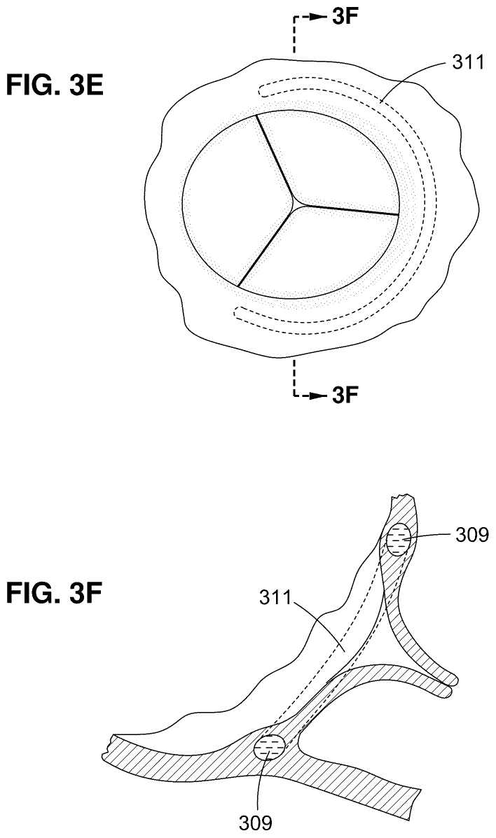

Provided in FIGS. 3A and 3B is an example of a catheter system for augmentation in which the distal end of the catheter that has been delivered to a regurgitant valve is shown. The system comprises a delivery catheter 301 and fluid injector or means for injection 303 for delivering and injecting a filler. In some implementations, fluid injector or means for injection 303 comprises a needle. In some implementations, fluid injector or means for injection 303 comprises a cannula. In some implementations, the fluid injector or means for injection 303 comprises one or more of a needle, a syringe, a cannula, a fluid line, a hypotube, a pump, a balloon, a hydraulic system, a power injector, a motor, etc. Fluid injector or means for injection 303 can include any of the features as described herein, including (but not limited to) a beveled and/or curved tip, a flexible shaft, a flexure joint, a steering system, pull wires, and/or means for active steerability.

In some implementations, delivery catheter 301 includes an inner sheath 305 that is in fluidic connection with fluid injector or means for injection 303. Inner sheath 305 can be utilized for the storage and transfer of filler. In some implementations, inner sheath 305 comprises a syringe or syringe-like system for expulsion of filler. In some implementations, inner sheath 305 is in fluidic connection with a pump for expulsion of filler.

FIGS. 3A to 3F depict an example of a method to augment an annulus with filler, the method utilizing the filler to expand and provide a pathway for the means of injection to be advance along the annulus.

At FIGS. 3A and 3B, delivery catheter 301 has been delivered to a regurgitant valve and fluid injector or means for injection 303 has been advanced distally out of an outer sheath 307. The distal end of fluid injector or means for injection 303 has inserted into the annulus of the valve, depositing an initial portion of filler 309 to expand the annulus near the injection path, creating a pathway 311. Fluid injector or means for injection 303 is distally advanced within pathway 311.

At FIG. 3C, fluid injector or means for injection 303 has distally advanced along pathway 311 about ⅓ of the circumference of the annulus. As fluid injector or means for injection 303 is distally advanced, filler is deposited to expand and augment the annulus, yielding pathway 311. The expansion and augmentation of the annulus also pushes on annular muscle toward the center of the valve, which in turn pushes the leaflet free edge towards the middle of valve, closing the gaps that allow regurgitation.

At FIG. 3D, fluid injector or means for injection 303 has distally advanced along pathway 311 about ⅔ of the circumference of the annulus. As fluid injector or means for injection 303 continues to be distally advanced, filler is deposited to expand and augment the annulus. The expansion and augmentation of the annulus results in the leaflets to better coapt and closing the gaps that allow regurgitation. Upon completion of depositing filler, fluid injector or means for injection 303 can be retracted proximately and out of the annulus. Additional filler can be deposited along another portion of the annulus and/or along a parallel track to already deposited filler.

At FIGS. 3E and 3F, delivery catheter 301 has been retracted and the results of annular augmentation is shown. Filler 309 has been deposited along pathway 311 to yield an augmented annulus that has improved the ability of the leaflets to coapt by closing the gaps that had allowed regurgitation.

While a specific configuration of a delivery catheter for augmentation is described above with reference to FIGS. 3A to 3F, it should be readily appreciated that various configurations of the system can be implemented in any of a variety of combinations of components. Accordingly, the specific configuration of the system described herein should be understood as not to be limited to any specific configuration, but instead can be implemented in any configuration capable of depositing filler via a delivery catheter. Likewise, the various steps of the method described above with reference to FIGS. 3A to 3F can all be included and/or some may be omitted. And additional steps not described here are also possible. Steps can be done in different orders as well.

Systems and devices for permanent (or semi-permanent) augmentation of the annulus comprise a catheter system such that it can be utilized within minimally invasive procedures and/or traverse through the circulatory system to reach the site of procedure. The systems and devices can comprise a delivery catheter for delivery of capsule and filler into the annulus of a valve. A delivery catheter systems and devices can comprise an inflator, expander, fluid injector, filler injector, or other means for expanding the capsule with filler into the annulus.

The systems and devices for permanent (or semi-permanent) augmentation can be utilized on the annulus of any of the four valves of the heart, including the mitral valve, the tricuspid valve, the pulmonary valve, and the aortic valve. Any transcatheter approach can be utilized to reach the valve to be treated, such as (for example) a transfemoral, subclavian, transapical, transjugular, or transaortic approach.

In some implementations, permanent (or semi-permanent) augmentation is achieved by expanding a capsule filled with filler in and around the annulus (or a portion thereof). In some implementations, the capsule is delivered in a compacted state to the annulus and then unfurled, elongated and filled. In some implementations, prior to elongation of the capsule, a track is tunneled through the annulus, which can be achieved using a steerable aspiration needle or any other device capable of tunneling a pathway through the annulus. In some implementations, a compacted capsule is powered to tunnel through and elongate along the annulus, which can be performed with or without a pre-tunneled track. In some implementations, the capsule is filled with a filler as the capsule is elongated. In some implementations, the capsule is filled with a filler after elongation of the capsule. In some instances, a pump is utilized to fill the capsule. In some instances, the pump powers (or assists) the tunneling of the capsule.

In some implementations, a catheter system for permanent (or semi-permanent) augmentation comprises a fluid injector or other means for filling the capsule with fluid. In some implementations, the inner catheter can comprise a means for manual filling. For example, in some implementations, the inner catheter can be a syringe (or is syringe-like) comprising a plunger for the expulsion of fluid through and out of the catheter into the capsule. Other means for manually pushing fluid out of an inner catheter can also be utilized. In some implementations, the inner catheter can be in connection with a pump that can provide pressurized fluid (e.g., filler, saline, carbon dioxide gas) to push the filler through and out the fluid injector or means for injection. In some implementations, the catheter system can comprise a pump, which can be provided within an outer sheath at or near the distal end of the catheter system or can be provided at the proximal end of the catheter system (e.g., extracorporeal). The pump can be in fluidic connection with the capsule such that it can pump filler into the capsule.

In some implementations, a catheter system for permanent (or semi-permanent) augmentation comprises a compacted capsule located at or near the distal end of the catheter system. In some implementations, a catheter system for permanent (or semi-permanent) augmentation comprises a removable nosecone to expose the compacted capsule. A compacted capsule can be rolled, folded, or compacted in any appropriate way. Upon removal of the nosecone, the pump can be utilized to unfurl and expand the capsule. The pump can also be utilized to fill the capsule with filler, which can be filled as it is expanded. After filling of the capsule with filler, the capsule can be tied off, capped, or otherwise sealed to yield a permanent (or semi-permanent) augmentation.

In some implementations, the capsule can elongate through at least a portion of the annulus. In various implementations, the capsule can elongate through at least ¾ of the annular circumference, the capsule can elongate through at least ⅔ of the annular circumference, the capsule can elongate through at least ½ of the annular circumference, the capsule can elongate through at least ⅓ of the annular circumference, the capsule can elongate through at least ¼ of the annular circumference, or the capsule can elongate through at least ⅛ of the annular circumference.

In some implementations, the pump is in fluidic connection with an inner catheter for the transfer of filler into the capsule. In some implementations, the inner catheter can be in connection with a source of filler, which can be kept at the proximal end of the catheter system.

Any biocompatible filler can be utilized for permanent annular augmentation. Examples of biocompatible fillers include (but are not limited to) silicone gel, saline, a solution comprising hyaluronic acid, a solution comprising collagen, a solution comprising elastin, a solution comprising donated fat, and combinations thereof. In one example, donated fat can be removed from a location within the patient's body (e.g., hips, thighs, abdomen) and transferred to the catheter system for injection. The choice of filler may depend, in part, on whether the augmentation is permanent or semi-permanent. Use of a permanent capsule allows for filler, such as silicone gel, that may be undesirable to be released in within the body. And when a semi-permanent capsule is utilized, filler that is more tolerable to dissipate within body, such as a solution of hyaluronic acid, can be utilized.

In some implementations, the capsule is a permanent biocompatible material. Permanent is to mean a material that is nonbiodegradable. Any biocompatible material capable of compact, elongating, and storing filler can be utilized for the capsule. Examples of biocompatible materials for a capsule include (but are not limited to) poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), and siloxanes.

In some implementations, the capsule is a semi-permanent biocompatible material. Semi-permanent is to mean a material that is also biodegradable over time, such that the capsule eventually breaks down and degrades within body. Any biocompatible material capable of compact, elongating, and semi-permanently storing filler can be utilized for the capsule. Examples of biocompatible and biodegradable material for use as a semi-permanent capsule include poly(lactic-co-glycolic acid) (PGLA), poly (β-hydroxybutyrate-co-β-hydroxy valerate) (PHBV), polyhydroxy butyrate (PHB), and polycaprolactone (PCL).

In some implementations, the delivery catheter system extends proximally to a control system, as is appreciated in the art of transcatheter procedures. A clinician can utilize the control system to advance the transcatheter system through the body to the site where the procedure is to be performed. Further, the control system can advance and retract the compacted capsule in the distal and proximal direction in reference to an outer sheath. In some implementations, the control system can be utilized to control the removal (and recapping) of the nosecone. In some implementations, the control system can further be utilized to control the filling of the capsule with filler.

When at the valve annulus, various augmentation procedures can be performed. In some situations, prior to unfurling and extending the compacted capsule, a tool is inserted at a location at or near the annulus to tunnel a path along annulus. The tool can be an aspiration needle or a core needle. The tool can be inserted and tunneled up to a desired amount creating a pathway. The tool can be retracted back through tissue, and then the capsule can be unfurled, elongated, and filled with filler such that the capsule elongates along the pathway. The procedure can be repeated as necessary such that the annulus (or a portion thereof) is augmented with encapsulated filler and the leaflets of the valve can better coapt.

In some situations, the elongation of capsule is performed without pretreating the annular tissue with a tool to create a pathway. Along the annulus there is fatty tissue that is adjacent to the fibrous annular tissue. Taking advantage of the softer fatty tissue that encircles the annulus, the capsule can be elongated through this tissue using the force provided by the pump to fill the capsule. To initiate the elongation, a tool can be used to create an incision or nick at a location at or near the fatty tissue at which point the capsule can begin to be elongated. The capsule can be further advanced through the fatty tissue by filling the capsule with filler using the pump. The capsule can be elongated up until a desired amount such that encircles at least a portion of the annulus. The procedure can be repeated as necessary such that the annulus (or a portion thereof) is augmented with encapsulated filler and the leaflets of the valve can better coapt.

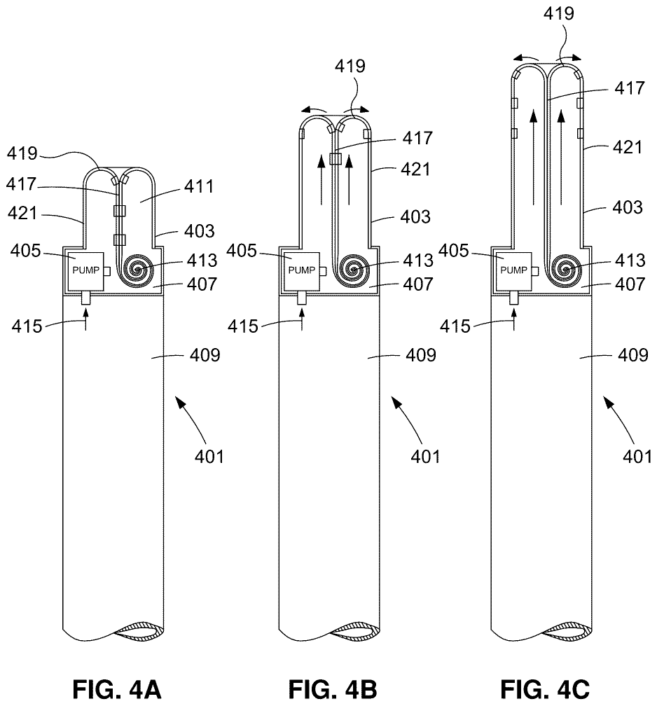

Provided in FIGS. 4A to 4C is an example of a catheter system utilizing a compacted capsule that is capable of being unfurled, expanded, and filled with a filler. Catheter system 401 comprises a capsule 403 that is compacted within the distal end of the catheter system. Capsule 403 is depicted as rolled, but it can be folded or compacted any way such that it fits within a catheter and is capable of being unfurled. Catheter system 401 can further comprise a fluid injector/filler injector or other means for filling the capsule with filler. As shown, catheter system 401 comprises a pump 405 for filling the capsule with filler, although other injection techniques can be utilized. Pump 405 is depicted as being within the distal portion of catheter system 401, but it can be located at a proximal portion of the catheter system as long as the pump is in fluidic connection with the capsule to deposit filler therein. Catheter system 401 further comprises an inner catheter 407 and an outer sheath 409 to contain capsule 403 therein as the catheter system traverses through the vasculature.

At FIG. 4A, to initiate the unfurling and extension of capsule 403, inner catheter 407 can be distally advanced to expose the capsule. A portion of capsule 403 can be pre-unfurled 411, and can further be pre-filled with filler to provide to help initiate the elongation. The remaining portion of capsule 403 is a compacted portion 413. A filler fluid 415 can be pumped into capsule 403 to further unfurl and elongate the capsule to form a tubular shape in with the central hollow area having an inner wall 417. Capsule 403 further has a distal wall 419 and an outer wall 421. To help show and explain the elongation mechanism and dynamic movement of the capsule walls, square markers are depicted, which are initially located on inner wall 417.

FIGS. 4B and 4C depicts capsule 403 further unfurled and elongated. Capsule 403 elongates in the tubular shape. Pumping in filler fluid 415 unfurls compacted portion 413 of capsule 403. As the compacted portion 413 unfurls and expands, it initially forms the inner wall 417 and then forms the distal wall 419 and then forms outer wall 421. During expansion, a circular “inner space” is formed within the luminal area formed by inner wall 417, but it is to be understood that the filling of capsule 403 can be expanded such that there is little to no inner space within the inner wall as the force of filling pushes the inner wall against itself.

As shown in FIG. 4B, as more of capsule 403 unfurls and expands, the first square marker has moved from inner wall 417 and past distal wall 419 onto outer wall 421. The other two square markers are still on inner wall 417 but have moved closer to distal wall 419.

As shown in FIG. 4C, as even more of capsule 403 unfurls and expands the first marker has remained in the same location on distal wall 419 onto outer wall 421. The other two markers have moved from inner wall 417 and past distal wall 419 onto outer wall 421. Capsule 403 continues to expand until it reaches a desired length. At that point, the proximal end of capsule 403 can be plugged, tied off, or otherwise sealed to yield an encapsulated filler for augmentation.

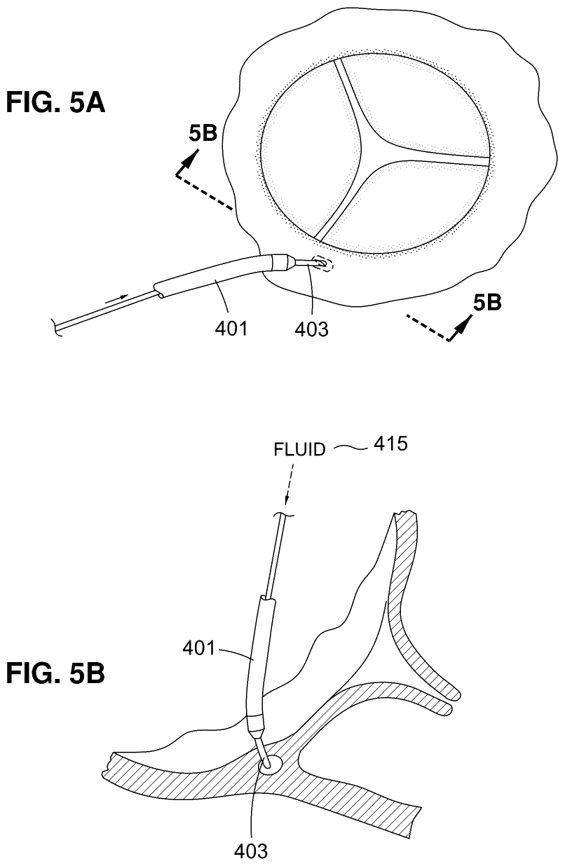

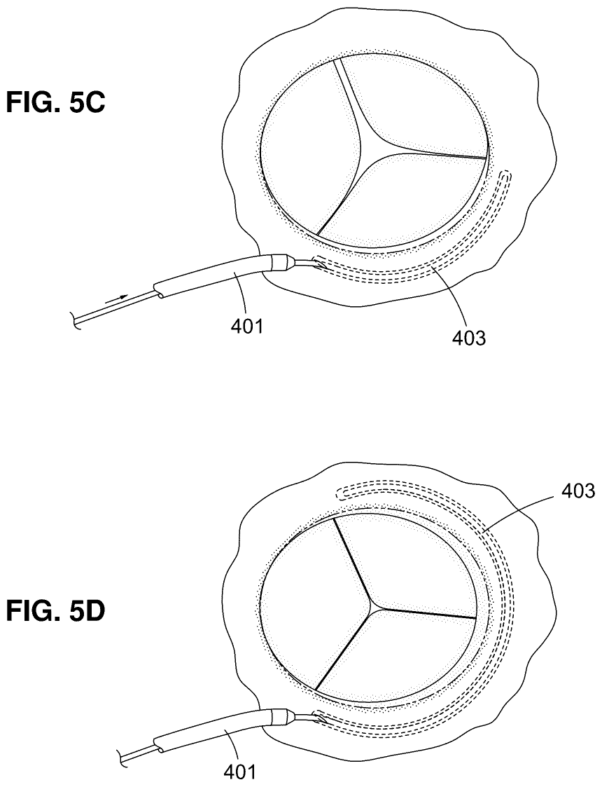

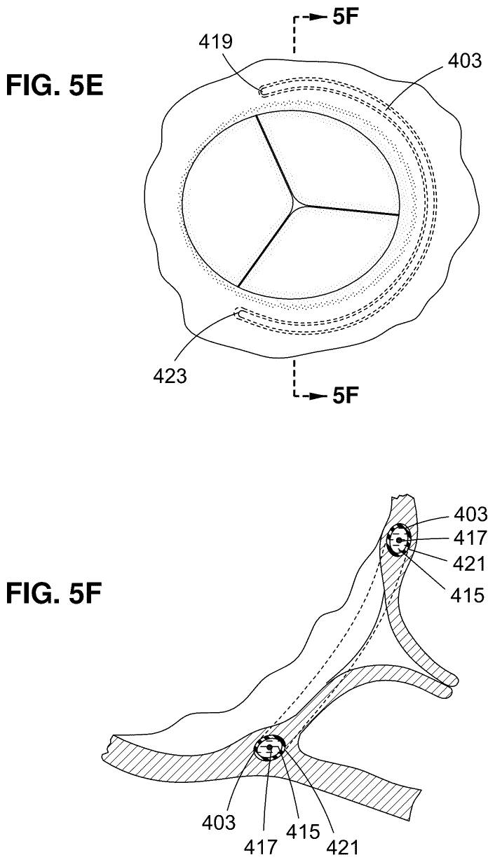

Provided in FIGS. 5A to 5F is an example method utilizing a catheter system to permanently or semi-permanently augment an annulus of a valve. The method utilizes the catheter system described in FIGS. 4A to 4C, but it should be understood that other catheter systems having similar design elements including a compacted capsule capable of unfurling and expanding can be utilized

Any transcatheter approach to reach the valve can be utilized, such as (for example) a transfemoral, subclavian, transapical, transjugular or transaortic approach.

As shown in FIGS. 5A and 5B, catheter system 401 has been translocated to the valve annulus and an initial portion of capsule 403 has been exposed and inserted into the annular tissue. In some implementations, a tool is utilized to create an incision or nick the annular tissue such that the distal wall of capsule 403 can be inserted into the tissue. In some implementations, a tool is utilized to create a track through the tissue along the circumference of the annulus.

With the distal tip inserted into the annular tissue, filler fluid is pumped into capsule 403, resulting in the compacted portion of the capsule to unfurl and elongate. At FIG. 5C, capsule 403 has elongated to about ⅓ of the annular circumference, augmenting the annular tissue. At FIG. 5D, capsule 403 has further elongated to about ⅔ of the annular circumference, further augmenting the annular tissue. The expansion and augmentation of the annulus also pushes on annular muscle toward the center of the valve, which in turn pushes the leaflet free edge towards the middle of valve, closing the gaps that allow regurgitation. At this point, the proximal end of capsule 403 is plugged, tied off, or sealed. Catheter system 401 can be removed, leaving the encapsulated filler within the valve annulus.

FIGS. 5E and 5F depict the result of the augmentation procedure. Capsule 403 has been elongated through the annular muscle, about ⅔ of the annular circumference. Capsule 403 is filled with filler fluid 415 within inner wall 417 and outer wall 421 and between distal wall 419 and sealed proximal wall 423. The encapsulated filler yields a permanent or semi-permanent augmented annulus that has improved the ability of the leaflets to coapt by closing the gaps that had allowed regurgitation.

While a specific configuration of a delivery catheter for permanent or semi-permanent augmentation is described above with reference to FIGS. 4A to 4C and FIGS. 5A to 5F, it should be readily appreciated that various configurations of the system can be implemented in any of a variety of combinations of components. Accordingly, the specific configuration of the system described herein should be understood as not to be limited to any specific configuration, but instead can be implemented in any configuration capable of expanding a capsule with filler via a delivery catheter. Likewise, the various steps of the method described above with reference to FIGS. 5A to 5F can all be included and/or some may be omitted. And additional steps not described here are also possible. Steps can be done in different orders as well.

Systems and devices for delivering an implant to the annulus comprise a catheter system such that it can be utilized within minimally invasive procedures and/or traverse through the circulatory system to reach the site of procedure. The systems and devices can comprise a delivery catheter for delivery and installing an implant at the annulus of a valve. Delivery catheter systems and devices can comprise an implant and any components for installing the implant. Further, catheter systems and devices can be utilized to excise annular tissue to provide an implantation site. Tissue excising catheter systems and devices can comprise a tissue excision tool, knife, cutting device, needle, and/or other means for excising tissue, and can be the same or different catheter system used for installing the implant.

The systems and devices for implant delivery can be utilized on the annulus of any of the four valves of the heart, including the mitral valve, the tricuspid valve, the pulmonary valve, and the aortic valve. Any transcatheter approach can be utilized to reach the valve to be treated, such as (for example) a transfemoral, subclavian, transapical, transjugular or transaortic approach.

A set of one or more implants can be installed. Each implant of the set of one or more implants can be strategically installed at particular locations within the annulus. Generally, each implant can be installed at a location within the annulus that would improve the ability of leaflets to coapt to mitigate regurgitation. Each implant can be installed at a location to push a nearby leaflet more towards the center of the valve. In some implementations, the set of one or more implants is installed on the lateral wall to prevent entanglement with conduction systems.

When the set of implants includes two or more, the set of implants can be strategically installed at locations around the annulus. In various implementations, the set of implants are installed at locations encircling the entire annular circumference, the set of implants are installed at locations encircling at least ¾ of the annular circumference, the set of implants are installed at locations encircling at least ⅔ of the annular circumference, the set of implants are installed at locations encircling at least ½ of the annular circumference, the set of implants are installed at locations encircling at least ⅓ of the annular circumference, the set of implants are installed at locations encircling at least ¼ of the annular circumference, or the set of implants are installed at locations encircling at least ⅛ of the annular circumference.

In some implementations, prior to installing an implant, a portion of annular tissue is excised. In some implementations, the tissue excision tool or means excision of annular tissue is performed using a sharp-edged tool delivered via a catheter system. In some implementations, the sharp-edged tool is a scalpel or a tissue shaver. In some implementations, the tissue excision tool or means excision of annular tissue is performed using a tissue ablation tool. A tissue ablation tool can excise tissue using heat ablation, radiofrequency ablation, cryoablation, or electroablation. Once tissue is excised or ablated, the tissue waste can be captured and removed. Various methods to remove tissue waste include (but are not limited to) grasping by a tool, aspiration or otherwise capturing within a tool, or vacuuming.

Excision of a portion of annular tissue provides a location to install an implant within the annulus, such that the implant can push a leaflet free edge towards the middle of valve, closing the gaps that allow regurgitation. Any type of implant that can provide bulk or otherwise provide push forces to bring leaflet free edges closer to better coapt can be utilized.

In some implementations, the implant is a biocompatible polymer capable of being delivered as a liquid or gel to be deposited within the annular tissue. In some implementations, the liquid or gel biocompatible polymer is capable of curing, hardening and/or solidifying upon or after depositing into the annular tissue. In some implementations, the hardening and/or solidifying of the liquid or gel biocompatible polymer can be performed by inducing crosslinking, which can be done by contacting with small crosslinking molecules, polymer-polymer conjugation, utilizing photosensitive agents and a source of light, and/or contacting with an enzyme to catalyze the crosslinking. In some implementations, the liquid or gel biocompatible polymer is a hydrogel. Hydrogels have the additional benefit that their size can be expanded utilizing water, saline, or other compounds, which may help provide push forces. Biocompatible polymers include natural and synthetic polymers. Examples of natural biocompatible polymers that can be utilized include (but are not limited to) hyaluronic acid, chitosan, heparin, alginate, gelatin, and fibrin. Examples of synthetic biocompatible polymers that can be utilized include (but are not limited to) poly(ethylene glycol) (PEG), poly(vinyl alcohol) (PVA), poly(acrylate), and poly(vinylpyrrolidone) (PVP).

In some implementations, the implant is a plurality of biocompatible beads. The plurality of biocompatible beads can be installed on its own or combined with liquid or gel biocompatible polymer. When combined with a liquid or gel biocompatible polymer, the beads can provide a more structural component within the implant. In some implementations, the biocompatible bead is hydrogel-based. Hydrogels have the additional benefit that their size can be expanded utilizing water, saline, or other compounds, which may help provide push forces. In some implementations, the biocompatible bead is silica-based. Biocompatible beads can be generated from biocompatible polymers and/or silica and may be formed, cured, hardened, and/or solidified prior to delivery. Biocompatible polymers include natural and synthetic polymers. Examples of natural biocompatible polymers that can be utilized include (but are not limited to) hyaluronic acid, chitosan, heparin, alginate, gelatin, and fibrin. Examples of synthetic biocompatible polymers that can be utilized include (but are not limited to) poly(ethylene glycol) (PEG), poly(vinyl alcohol) (PVA), poly(acrylate), and poly(vinylpyrrolidone) (PVP).

In some implementations, the implant is an embolization coil, such as those used for aneurysms. A catheter system can deliver the embolization coil to the site of installation and deposit therein where it can fill and expand within the annular tissue. Any biocompatible metal with linear elasticity can be utilized. Materials used for coiled embolization include (but are not limited to) stainless steel, platinum, nickel, cobalt, titanium, and alloys thereof.

In some implementations, the implant is an expandable stent. A catheter system can deliver the stent to the site of installation and install therein where it can expand within the annular tissue. In some implementations, the stent includes an impermeable (or substantially impermeable) cover that is capable of expanding across the installation site to seal the cavity created by the tissue excision. The use of a cover inhibits and/or prevents blood flow into the formed cavity. In some implementations, the stent is a self-expanding stent, such as stents composed of shape-memory nitinol. In some implementations, the stent can be expanded using an expander (e.g., balloon). Materials used for stents include (but are not limited to) stainless steel, platinum, nickel, cobalt, titanium, and alloys thereof. Materials used for covers include (but are not limited to) silk fibroin (SF), chitosan, poly(ethylene) (PE), poly(ethylene terephthalate) (PET), ultra-high molecular weight poly(ethylene) (UHMWPE), thermoplastic poly(urethane) (TPU), expanded poly(tetrafluoroethylene) (ePTFE), poly(caprolactone) (PCL), poly(vinylpyrrolidone) (PVP), and siloxanes; which can be combined and/or fluorinated.

In some implementations, the implant delivery catheter system extends proximally to a control system, as is appreciated in the art of transcatheter procedures. A clinician can utilize the control system to advance the transcatheter system through the body to the site where the procedure is to be performed. Further, the control system can advance and retract the implant, a sheath, a wire, a catheter, etc. and/or a means for installing an implant. In some implementations, the control system can advance and retract the tissue excision tool or means for excising and removing tissue. In some implementations, the control system can further be utilized to control the injection of filler.

In some situations, the tissue excision tool or means for excising tissue is inserted at a location at or near the annulus. In some situations, in which a plurality of implants is to be installed, the tissue excision tool or means for excising tissue is inserted at each location along the annulus that an implant is to be installed. In some implementations, the catheter system is configured to perform each excision with a single approach. In some instances, the catheter system can approach the annulus multiple times to excise tissue, retracting back proximally after each approach with excised tissue. In some instances, an aspiration line is provided within the catheter system to aspirate and remove excised tissue.

Once tissue has been excised, an implant can be installed in the cavity where the tissue has been excised. In some implementations, the catheter system is configured to perform one or more excisions and one or more implant installations in a single approach. In some implementations, the catheter system is configured to perform all excisions and all implant installations in a single approach. In some implementations, the catheter system is configured to perform one or more excisions in a single approach and then perform one or more implant installations in a subsequent approach. In some implementations, the catheter system is configured to perform all excisions in a single approach and then perform all implant installations in a subsequent approach.

In some implementations, the set of one or more cavities is sealed with the implant installed inside. Any biocompatible material capable providing a seal can be utilized. In some implantations, liquid or gel biocompatible polymer is deposited and cured. In some implantations, a biocompatible adhesive or glue is deposited. In some instances, compounds for reendothelialization are deposited to promote native tissue growth over the cavity. In some implementations, a reendothelialization-inducing biologic is deposited, such as (for example) amino acids (e.g., lysine or ornithine), saccharides (e.g., hyaluronic acid, fibronectin, chitosan), structural proteins (e.g., collagen, elastin), growth factors (e.g., VEGF), and combinations thereof.

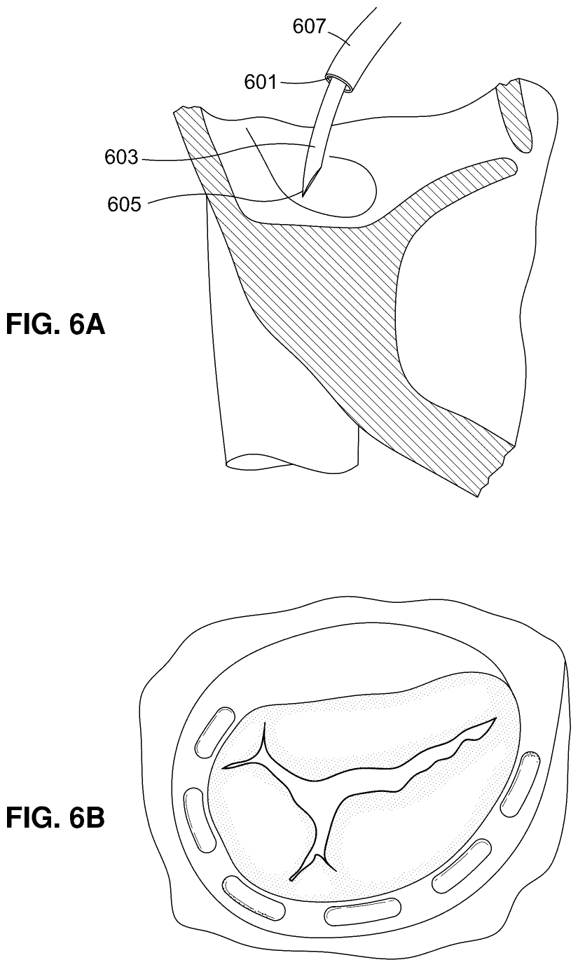

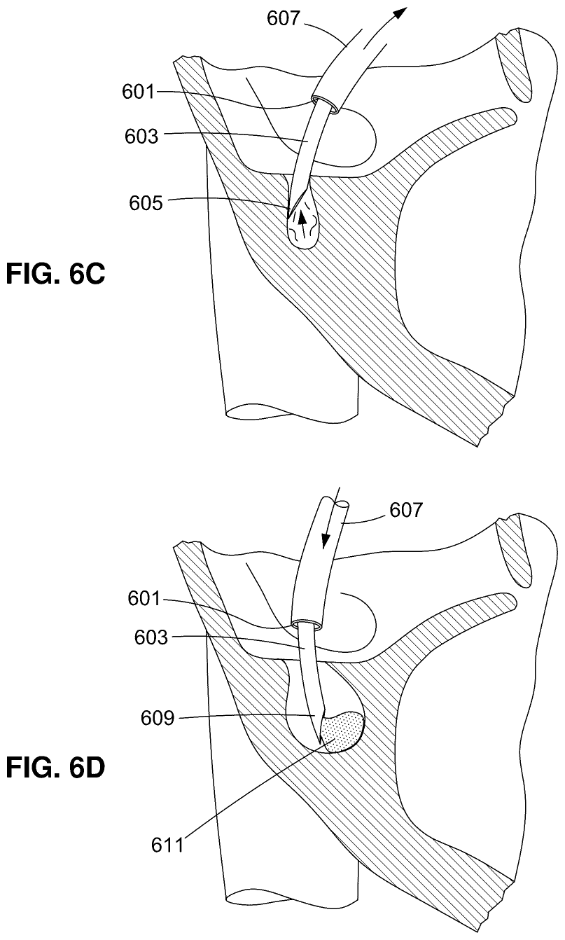

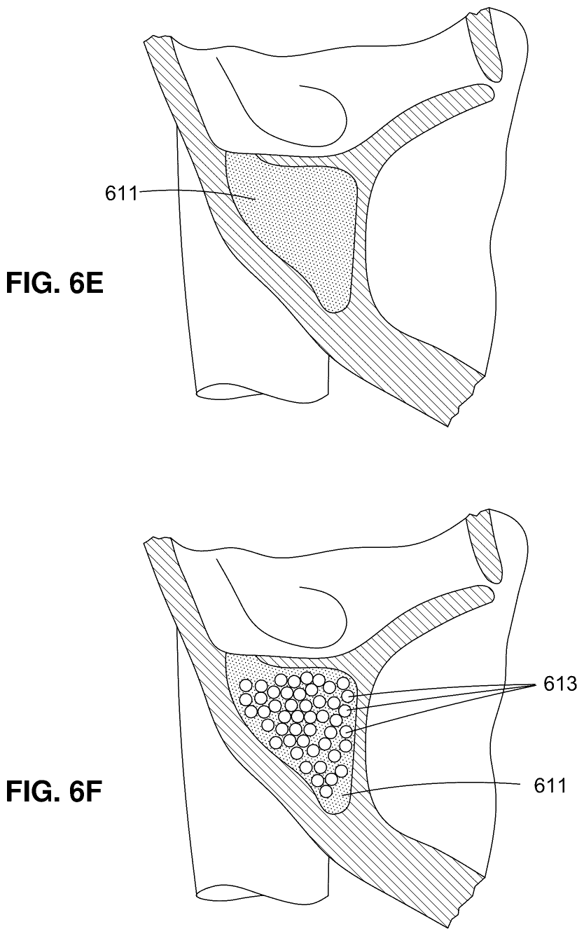

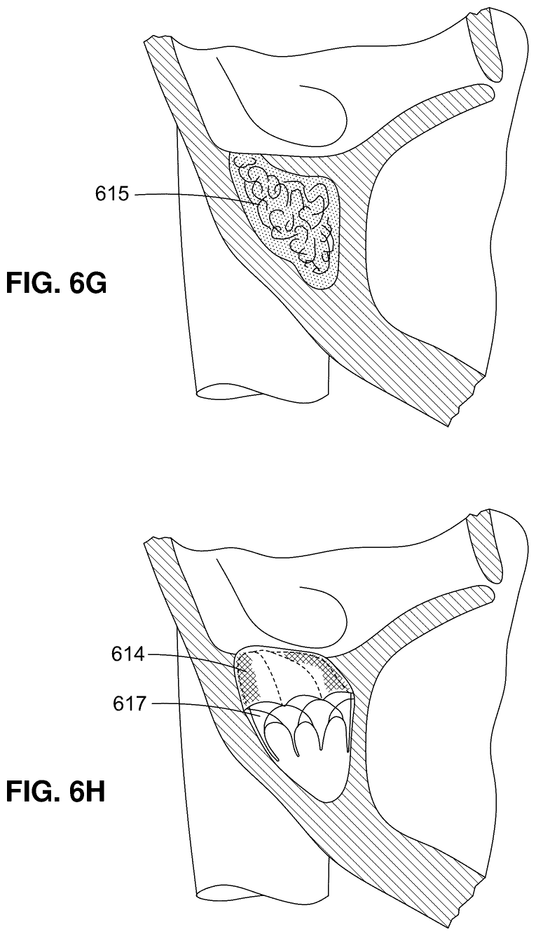

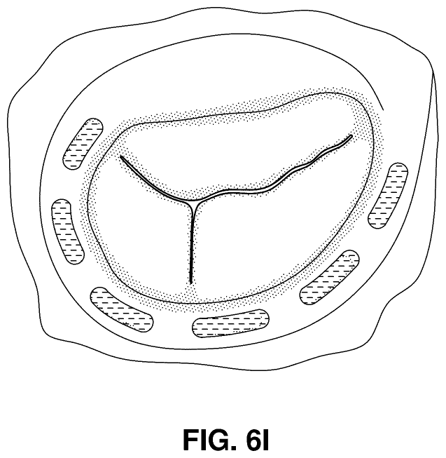

Provided in FIGS. 6A to 6I is an example of a catheter system for installing an annular implant in which the distal end of the catheter that has been delivered to a regurgitant valve is shown. The system comprises a catheter 601, which can provide a tissue excision tool or means for tissue excision and/or a delivery system or means for implant delivery. In some implementations, catheter 601 includes an inner sheath 603 that provides an ability to distally extend and proximally retract the tissue excision tool/means for tissue excision and/or the delivery system/means for implant delivery. Tissue can be excised utilizing a sharp edge, knife, needle, etc., an ablation technique, aspiration technique, or any combination thereof. Implants can be deposited and/or installed using catheter 601 (e.g., as part of the delivery system or means for delivery), as would be dependent on the type of implant.

FIGS. 6A to 6I also depict examples of a method to install various implants within an annulus. The method generally includes steps of excising annular tissue and then installing an implant within the cavity created by the tissue excision, and expanding the annular area to provide annular push forces to help the native leaflets coapt.

At FIG. 6A, catheter 601 has been delivered to the annulus of a regurgitant valve and an incision tool/excision tool or means for incising/excising tissue 605 has been advanced distally out of an outer sheath 607 via the inner sheath 603. The distal end of the incision/excision tool or means for incising tissue 605 comes into contact with the annular tissue.

At FIG. 6C, incision tool/excision tool or means for incising/excising tissue 605 has created a cavity. In some implementations, the tissue waste is vacuumed or aspirated, as depicted. In some implementations, the tissue waste is grasped for removal. In some implementations, a set of one or more cavities are created. As an example, in FIG. 6B, six cavities are created. In some implementations, only one cavity is created. In some implementations, the set of one or more cavities is adjacent to the lateral wall of the heart.

At FIG. 6D, catheter 601 extends the distal end of a delivery system, catheter, or other means for delivering an implant 609. As depicted, the delivery system, catheter, or other means for delivering an implant is depositing a liquid or gel biocompatible polymer 611 into the cavity. FIG. 6E depicts a cavity that has been filled with a liquid or gel biocompatible polymer 611. FIG. 6F depicts a cavity that has been filled with biocompatible beads 613 and a liquid or gel biocompatible polymer 611. FIG. 6G depicts a cavity that has been filled with an embolization coil 615. FIG. 6H depicts a cavity that has a stent 617 installed therein. Stent 617 comprises a cover 619 that expands across an opening of the stent such that expands across the opening of the cavity. FIG. 6I depicts each cavity of the set of cavities having an implant installed therein. Each cavity can also be sealed. Installation of the various implants can result in expansion of the annular region to yield push forces that help coapt the leaflets.

While a specific configuration of a delivery catheter for implant delivery is described above with reference to FIGS. 6A to 6I, it should be readily appreciated that various configurations of the system can be implemented in any of a variety of combinations of components. Accordingly, the specific configuration of the system described herein should be understood as not to be limited to any specific configuration, but instead can be implemented in any configuration capable of installing an implant via a delivery catheter. Likewise, the various steps of the method described above with reference to FIGS. 6A to 6I can all be included and/or some may be omitted. And additional steps not described here are also possible. Steps can be done in different orders as well.

### EXAMPLES

Example 1. A system for augmenting tissue, comprising:
a transcatheter system, wherein the transcatheter system comprises an outer sheath and a fluid injector or other means for fluid injection in fluidic connection with a biocompatible filler fluid;
wherein the fluid injector or other means for fluid injection is capable of being distally advanced and proximately retracted in reference to the outer sheath.
Example 2. The system of example 1, wherein the fluid injector or other means for fluid injection comprises at least one of a needle and/or a cannula.
Example 3. The system of any one of examples 1-2, wherein the fluid injector or other means for fluid injection is flexible.
Example 4. The system of any one of examples 1-3, wherein the fluid injector or other means for fluid injection has a tip that is beveled, curved, or kinked.
Example 5. The system of any one of examples 1-4, wherein the biocompatible filler fluid comprises one of: saline, a solution comprising hyaluronic acid, a solution comprising collagen, a solution comprising elastin, or a solution comprising donated fat.
Example 6. A method for augmenting annular tissue of a valve, comprising:
advancing a transcatheter system to an annulus of a heart valve, wherein the transcatheter system comprises an outer sheath and a fluid injector or other means for fluid injection in fluidic connection with a biocompatible filler fluid, wherein the means for fluid injection comprises at least one of a needle and/or a cannula; and
injecting the biocompatible filler fluid into the annulus.
Example 7. The method of example 6, wherein the step of injecting the biocompatible filler fluid comprises:
repeatedly inserting the means of injection at a plurality of injection sites along the annulus; and
depositing the biocompatible filler fluid at each of the plurality of injection sites.
Example 8. The method of example 7, wherein the fluid injector or other means for injection is flexible and capable of tunneling within and around the annulus.
Example 9. The method of example 8, wherein the step of injecting the biocompatible filler fluid comprises:
inserting the fluid injector or other means of injection within the annulus;
tunneling the fluid injector or other means of injection through and around the annulus; and
retracting the fluid injector or other means of injection back through and around the annulus, depositing the biocompatible filler fluid as the fluid injector or other means of injection is being retracted.
Example 10. The method of example 8, wherein the step of injecting the biocompatible filler fluid comprises:
inserting the fluid injector or other means of injection within the annulus;
depositing a portion of the biocompatible filler fluid at or near the insertion site; and
advancing the fluid injector or other means of injection through and around the annulus, depositing the biocompatible filler fluid as the means of injection is advanced.
Example 11. A system for permanently or semi-permanently augmenting tissue, comprising:
a transcatheter system, wherein the transcatheter system comprises an outer sheath and a compacted capsule in fluidic connection with a pump, wherein the compacted capsule is capable of being unfurled and elongated by filling the capsule with a filler;
wherein the compacted capsule is at or near a distal end of the transcatheter system within the outer sheath.
Example 12. The system of example 11, wherein the transcatheter system further comprises a tool for creating an incision or nick in tissue, wherein the tool is at or near a distal end of the transcatheter system within the outer sheath.
Example 13. The system of any one of examples 11-12, wherein the capsule comprises a nonbiodegradable biocompatible material.
Example 14. The system of any one of examples 11-13, wherein the capsule comprises at least one of the following materials: poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), or a siloxane.
Example 15. The system of any one of examples 11-14, wherein the capsule comprises a biodegradable biocompatible material.
Example 16. The system of any one of examples 11-15, wherein the capsule comprises at least one of the following materials: poly(lactic-co-glycolic acid) (PGLA), poly (β-hydroxybutyrate-co-β-hydroxy valerate) (PHBV), polyhydroxy butyrate (PHB), or polycaprolactone (PCL).
Example 17. The system of any one of examples 11-16, wherein the filler comprises:
silicone gel, saline, a solution comprising hyaluronic acid, a solution comprising collagen, a solution comprising elastin, or a solution comprising donated fat.
Example 18. The system of any one of examples 11-17, wherein the transcatheter system further comprises a removable nosecone at the distal end of the transcatheter system.
Example 19. The system of any one of examples 11-18, wherein the pump is located within an outer catheter.
Example 20. The system of any one of examples 11-18, wherein the pump is located at a proximal end of the transcatheter system.
Example 21. A method for augmenting annular tissue of a valve, comprising:
advancing a transcatheter system to an annulus of a heart valve, wherein the transcatheter system comprises a capsule that is compacted and is in fluidic connection with a pump;
creating an incision within the annulus; and
elongating the capsule within the incision and through and around the annulus by filling the capsule with a filler via the pump.
Example 22. The method of example 21, wherein the step of elongating the capsule comprises elongating the capsule through softer fatty tissue that encircles the annulus.
Example 23. The method of any one of examples 21-22 further comprising:
prior to elongating the capsule, inserting a tool within the incision and advancing the tool through and around the annulus to create a track.
Example 24. The method of any one of examples 21-23, wherein the capsule comprises a nonbiodegradable biocompatible material.
Example 25. The method of any one of examples 21-24, wherein the capsule comprises at least one of the following materials: poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), or a siloxane.
Example 26. The method of any one of examples 21-25, wherein the capsule comprises a biodegradable biocompatible material.
Example 27. The method of any one of examples 21-26, wherein the capsule comprises at least one of the following materials: poly(lactic-co-glycolic acid) (PGLA), poly (β-hydroxybutyrate-co-β-hydroxy valerate) (PHBV), polyhydroxy butyrate (PHB), or polycaprolactone (PCL).
Example 28. The method of any one of examples 21-27, wherein the filler comprises:
silicone gel, saline, a solution comprising hyaluronic acid, a solution comprising collagen, a solution comprising elastin, or a solution comprising donated fat.
Example 29. The method of any one of examples 21-28, wherein the step of elongating the capsule comprises elongating the capsule through at least ⅛ of the annular circumference.
Example 30. The method of any one of examples 21-29 further comprising: sealing the capsule.
Example 31. A system for installing an implant in tissue, comprising:
a transcatheter system, wherein the transcatheter system comprises a tissue excision tool or other means for excising tissue, an implant, and a delivery tool or other means for installing the implant within tissue.
Example 32. The system of example 31, wherein the tissue excision tool or other means for excising tissue is a sharp-edged tool.
Example 33. The system of example 32, wherein the sharp-edged tool is a scalpel or a tissue shaver.
Example 34. The system of any one of examples 31-33, wherein the tissue excision tool or other means for excising tissue is a tissue ablation tool.
Example 35. The system of example 34, wherein the tissue ablation tool uses heat ablation, radiofrequency ablation, cryoablation, or electroablation.
Example 36. The system of any one of examples 31-35, wherein the implant comprises a biocompatible polymer that is capable of being delivered as a liquid or gel.
Example 37. The system of example 36, wherein the biocompatible polymer is capable of curing, hardening, or solidifying.
Example 38. The system of any one of examples 36-37, wherein the biocompatible polymer is a hydrogel.
Example 39. The system of any one of examples 31-38, wherein the implant comprises a plurality of biocompatible beads.
Example 40. The system of example 39, wherein each biocompatible bead is a silica-based or hydrogel-based.
Example 41. The system of any one of examples 31-40, wherein the implant comprises an embolization coil.
Example 42. The system of example 41, wherein the embolization coil comprises a biocompatible metal with linear elasticity.
Example 43. The system of any one of examples 31-42, wherein the implant comprises an expandable stent.
Example 44. The system of example 43, wherein the expandable stent is self-expanding.
Example 45. The system of any one of examples 43-44, wherein the expandable stent comprises a substantially impermeable cover.
Example 46. A method for installing an implant in an annulus of a heart valve, comprising: advancing a transcatheter system to an annulus of a heart valve, wherein the transcatheter system comprises a tissue excision tool or other means for excising tissue, an implant, and a delivery tool or other means for installing the implant within tissue;
excising tissue, using the tissue excision tool or other means for excising tissue, from the annulus to form a cavity therein; and
installing the implant in the cavity.
Example 47. The method of example 46, wherein the tissue excision tool or other means for excising tissue is a sharp-edged tool.
Example 48. The method of example 47, wherein the sharp-edged tool is a scalpel or a tissue shaver.
Example 49. The method of any one of examples 46-48, wherein the tissue excision tool or other means for excising tissue is a tissue ablation tool.
Example 50. The method of example 49, wherein the tissue ablation tool uses heat ablation, radiofrequency ablation, cryoablation, or electroablation.
Example 51. The method of any one of examples 46-50, wherein the implant comprises a biocompatible polymer capable of being delivered as a liquid or gel, and wherein the step of installing the implant comprises depositing the biocompatible polymer into the cavity.
Example 52. The method of example 51, wherein the biocompatible polymer is capable of curing, hardening, or solidifying.
Example 53. The method of any one of examples 51-52, wherein the biocompatible polymer is a hydrogel.
Example 54. The method of any one of examples 46-53, wherein the implant comprises a plurality of biocompatible beads, and wherein the step of installing the implant comprises depositing the plurality of biocompatible beads into the cavity.
Example 55. The method of example 54, wherein each biocompatible bead is a silica-based or hydrogel-based.
Example 56. The method of any one of examples 46-55, wherein the implant comprises an embolization coil and wherein the step of installing the implant comprises depositing the embolization coil into the cavity.
Example 57. The method of example 56, wherein the embolization coil comprises a biocompatible metal with linear elasticity.
Example 58. The method of any one of examples 46-57, wherein the implant comprises an expandable stent, and wherein the step of installing the implant comprises expanding the expandable stent within the cavity.
Example 59. The method of example 58, wherein the expandable stent is self-expanding.
Example 60. The method of any one of examples 58-59, wherein the expandable stent comprises a substantially impermeable cover.

What is claimed is:

1. A method for augmenting annular tissue of a valve, comprising:
advancing a transcatheter system to an annulus of a heart valve, wherein the transcatheter system comprises an outer sheath and a fluid injector or other means for fluid injection in fluidic connection with a biocompatible filler fluid, wherein the means for fluid injection comprises at least one of a needle and/or a cannula; and
injecting the biocompatible filler fluid into the annulus.

2. The method of claim 1, wherein the step of injecting the biocompatible filler fluid comprises:
repeatedly inserting the means of injection at a plurality of injection sites along the annulus; and
depositing the biocompatible filler fluid at each of the plurality of injection sites.

3. The method of claim 2, wherein the fluid injector or other means for injection is flexible and capable of tunneling within and around the annulus.

4. The method of claim 3, wherein the step of injecting the biocompatible filler fluid comprises:
inserting the fluid injector or other means of injection within the annulus;
tunneling the fluid injector or other means of injection through and around the annulus; and
retracting the fluid injector or other means of injection back through and around the annulus, depositing the biocompatible filler fluid as the fluid injector or other means of injection is being retracted.

5. The method of claim 3, wherein the step of injecting the biocompatible filler fluid comprises:
inserting the fluid injector or other means of injection within the annulus;
depositing a portion of the biocompatible filler fluid at or near the insertion site; and
advancing the fluid injector or other means of injection through and around the annulus, depositing the biocompatible filler fluid as the means of injection is advanced.

6. A system for permanently or semi-permanently augmenting tissue, comprising:
a transcatheter system, wherein the transcatheter system comprises an outer sheath and a compacted capsule in fluidic connection with a pump, wherein the compacted capsule is capable of being unfurled and elongated by filling the capsule with a filler;
wherein the compacted capsule is at or near a distal end of the transcatheter system within the outer sheath.

7. The system of claim 6, wherein the transcatheter system further comprises a tool for creating an incision or nick in tissue, wherein the tool is at or near a distal end of the transcatheter system within the outer sheath.

8. The system of claim 6, wherein the capsule comprises at least one of the following materials: poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), or a siloxane.

9. The system of claim 6, wherein the capsule comprises at least one of the following materials: poly(lactic-co-glycolic acid) (PGLA), poly (β-hydroxybutyrate-co-β-hydroxy valerate) (PHBV), polyhydroxy butyrate (PHB), or polycaprolactone (PCL).

10. The system of claim 6, wherein the filler comprises: silicone gel, saline, a solution comprising hyaluronic acid, a solution comprising collagen, a solution comprising elastin, or a solution comprising donated fat.

11. The system of claim 6, wherein the transcatheter system further comprises a removable nosecone at the distal end of the transcatheter system.

12. A method for augmenting annular tissue of a valve, comprising:
advancing a transcatheter system to an annulus of a heart valve, wherein the transcatheter system comprises a capsule that is compacted and is in fluidic connection with a pump;
creating an incision within the annulus; and
elongating the capsule within the incision and through and around the annulus by filling the capsule with a filler via the pump.

13. The method of claim 12, wherein the step of elongating the capsule comprises elongating the capsule through softer fatty tissue that encircles the annulus.

14. The method of claim 12 further comprising:
prior to elongating the capsule, inserting a tool within the incision and advancing the tool through and around the annulus to create a track.

15. The method of claim 12, wherein the capsule comprises at least one of the following materials: poly(dimethyl siloxane) PDMS elastomer, poly(ethylene (PE), poly(ethylene terephthalate) (PET), ultra-high molecular weight polyethylene (UHMWPE), thermoplastic poly(urethane (TPU), expanded poly(tetrafluoroethylene) (ePTFE), poly(caprolactone) (PCL), poly(vinylpyrrolidone) (PVP), or a siloxane.

16. The method of claim 12, wherein the capsule comprises a biodegradable biocompatible material.

17. The method of claim 16, wherein the capsule comprises at least one of the following materials: poly(lactic-co-glycolic acid) (PGLA), poly (β-hydroxybutyrate-co-β-hydroxy valerate) (PHBV), polyhydroxy butyrate (PHB), or polycaprolactone (PCL).

18. The method of claim 12, wherein the filler comprises: silicone gel, saline, a solution comprising hyaluronic acid, a solution comprising collagen, a solution comprising elastin, or a solution comprising donated fat.

19. The method of claim 12, wherein the step of elongating the capsule comprises elongating the capsule through at least ⅛ of the annular circumference.

20. The method of claim 12 further comprising: sealing the capsule.
